(19) United States
(12) Patent Application Publication
(10) Pub. No.: US 2026/0053837 A1
(43) Pub. Date: Feb. 26, 2026

(54) NICOTINAMIDE-RIBOSIDE AND PTEROSTILBENE COMBINATIONS TO TREAT OSTEOARTHRITIS

(71) Applicants: The Regents of the University of California, Oakland, CA (US); University of South Alabama, Mobile, AL (US)

(72) Inventors: Ru Liu Bryan, San Diego, CA (US); Marie Migaud, Mobile, AL (US)

(21) Appl. No.: 19/105,667

(22) PCT Filed: Sep. 1, 2023
(86) PCT No.: PCT/US2023/031900

§ 371 Date: Feb. 21, 2025

Related U.S. Application Data
(60) Provisional application No. 63/374,521, filed on Sep. 2, 2022.

(51) Int. Cl. A61K 31/706; A61K 31/09; A61P 19/02

(52) U.S. Cl. CPC A61K 31/706; A61K 31/09; A61P 19/02

(57) ABSTRACT

Compositions and methods of treatment for osteoarthritis and cartilage damage in a subject comprising administering to a subject in need thereof an effective amount of a nicotinamide-riboside (NR) and pterostilbene (PT) in a ratio of about 2:1.

### CROSS REFERENCE TO RELATED APPLICATIONS

The present application claims the priority benefit of U.S. Provisional Application No. 63/374,521 filed on Sep. 2, 2022, the entire contents of which are incorporated herein by reference.

### TECHNICAL FIELD

The present invention relates to treatments for osteoarthritis. The present invention relates to treatments for osteoarthritis

### BACKGROUND

Joint injury and aging are the primary risk factors for development of osteoarthritis (OA), the most common form of arthritis. Currently, there are not yet effective medical therapies to prevent or slow the disease process in OA. Thus, developing effective OA disease-modifying medical therapy is a major unmet medical need. Progressive degeneration of articular cartilage is a core OA disease feature. Chondrocytes, the sole cells in articular cartilage, are responsible for maintaining the homeostatic balance between extracellular matrix anabolism and catabolismin OA, chondrocytes undergo metabolic alterations triggering changes in energy metabolism due to altered bioenergy sensing, leading to chondrocyte dysfunction. This is amplified by local inflammatory processes, which promote excessive chondrocyte catabolic responses by inducing expression and activities of matrix degrading enzymes such as matrix metalloproteinases (MMPs).

Nicotinamide adenine dinucleotide (NAD⁺), a key intermediate metabolite, serves as a cofactor for numerous enzymes involved in cellular energy metabolism. It modulates adaptive responses of cells to bioenergetic and oxidative stress, and critically regulates multiple cell-signaling pathways. Mounting evidence indicates that maintenance of intracellular NAD⁺ content is critical for tissue homeostasis. Changes in NAD⁺ levels are related to the balance between NAD⁺ biosynthesis and degradation. Most cellular NAD⁺ is synthesized through salvage pathways from nicotinamide (NAM) or from NAD⁺ precursors (e.g., nicotinamide riboside (NR)). Another NAD⁺ precursor dihydronicotinamide riboside (NRH), a reduced form of NR, uses a newly identified pathway for NAD biosynthesis. Cellular NAD⁺ levels decline with aging, associated with increased expression of CD38, the main NADase in mammalian tissues contributing substantially to NAD degradation. Inflammation can cause NAD⁺ decline due to activation of consumers of NAD⁺ such as CD38.

Nicotinamide (NR) is a NAD precursor that can promote NAD biosynthesis. Pterostilbene (PT) is a potent activator of SIRT1, NAD-dependent protein deacetylase. SIRT1 that has been shown to play an important role in cartilage homeostasis. SIRT1 deficiency in chondrocytes is linked to OA development.

Elysium Health, Inc. provides an oral Nicotinamide-riboside (NR) and pterostilbene (PT) formulation (NR: PT in a 5:1 ratio, commercially marketed as “Basis”) which has been shown to increase NAD⁺ in humans and be safe for prolonged use.

### SUMMARY OF THE INVENTION

The disclosure provides methods of treatment, including prevention, of osteoarthritis, cartilage injury and cartilage degradation in a subject comprising administering to a subject in need thereof an effective amount of a composition comprising nicotinamide-riboside (NR) and pterostilbene (PT) in a ratio of between about 4:1 and 1:1, or between about 3:1 and 1.5:1, or about 2:1. In embodiments, the NRH: PT ratio is about 2:1.

In embodiments, the invention provides that the administration decreases nitric oxide (NO) levels in articular chondrocytes of the subject.

In embodiments, the invention provides that the composition further comprises pharmaceutically acceptable excipient.

In embodiments, the invention provides that the administration inhibits progression of osteoarthritis.

In embodiments, the invention provides that the administration is oral or parenteral.

In embodiments, the invention provides pharmaceutical compositions comprising composition comprising nicotinamide-riboside (NR) and pterostilbene (PT) in a ratio of between about 4:1 and 1:1, or between about 3:1 and 1.5:1, or about 2:1.

In embodiments, the disclosure provides methods of treatment, including prevention, of osteoarthritis, cartilage injury and cartilage degradation in a subject comprising co-administering to a subject in need thereof an effective amount of nicotinamide-riboside (NR) and pterostilbene (PT) in a ratio of between about 4:1 and 1:1, or between about 3:1 and 1.5:1, or about 2:1. In embodiments, the NRH: PT ratio is about 2:1. In embodiments the co-administration is “non-fixed combination” means that the NR and the PT are both administered to a patient as separate entities either simultaneously, concurrently or sequentially within 1-6 hours.

### BRIEF DESCRIPTION OF THE DRAWINGS

FIG. 1. Effects of various combination of NR and PT on NO release in human chondrocytes stimulated with IL-1b.

FIG. 2. Mice received NR, PT or NR+PT all experienced less pain in an injury-induced OA model.

FIG. 3 shows Blood NAD Metabolome tracing of isotopically labeled NR MW+8, ¹³C 5 ribose, ¹⁸O (amide), ¹⁵N (pyridine)) NR with and without PT.

FIG. 4 shows Blood NAD metabolites ratio of naturally occurring NAD, and NADH change in favor of NAD when NR is combined to PT.

FIG. 5 shows Joint Tissue NAD Metabolites tracing of isotopically labeled NR MW+8, ¹³C 5 ribose, ¹⁸O (amide), ¹⁵N (pyridine)) NR with and without PT.

FIG. 6 shows further Joint Tissue NAD Metabolites tracing of isotopically labeled NR MW+8, ¹³C 5 ribose, ¹⁸O (amide), ¹⁵N (pyridine)) NR with and without PT.

### DETAILED DESCRIPTION

The present disclosure relates, in part, to compositions and methods of treatment for osteoarthritis, cartilage injury, cartilage degradation, and related conditions.

The disclosure provides methods of treatment, including prevention, of osteoarthritis and cartilage injury and cartilage degradation in a subject comprising administering to a subject in need thereof an effective amount of a composition comprising nicotinamide-riboside (NR) and pterostilbene (PT) in a ratio of between about 4:1 and 1:1, or between about 3:1 and 1.5:1, or about 2:1. In embodiments, the NRH: PT ratio is about 2:1.

In embodiments, the invention provides that the administration decreases nitric oxide (NO) levels in articular chondrocytes of the subject.

In embodiments, the invention provides that the composition further comprises pharmaceutically acceptable excipient.

In embodiments, the invention provides that the administration inhibits progression of osteoarthritis.

In embodiments, the invention provides that the administration is oral or parenteral.

In embodiments, the invention provides pharmaceutical compositions comprising composition comprising nicotinamide-riboside (NR) and pterostilbene (PT) in a ratio of between about 4:1 and 1:1, or between about 3:1 and 1.5:1, or about 2:1.

In embodiments, the disclosure provides methods of treatment, including prevention, of osteoarthritis, cartilage injury and cartilage degradation in a subject comprising co-administering to a subject in need thereof an effective amount of nicotinamide-riboside (NR) and pterostilbene (PT) in a ratio of between about 4:1 and 1:1, or between about 3:1 and 1.5:1, or about 2:1. In embodiments, the NRH: PT ratio is about 2:1. In embodiments the co-administration is “non-fixed combination” means that the NR and PT are both administered to a patient as separate entities either simultaneously, concurrently or sequentially within 1-6 hours.

Various further aspects and embodiments of the disclosure will be apparent to one of ordinary skill in view of following description and examples.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention may employ conventional techniques of molecular biology (including recombinant techniques), microbiology, cell biology, biochemistry and immunology, which are within the skill of the art. Such techniques are explained fully in the literature, such as Methods in Molecular Biology, Humana Press: Cell Biology: A Laboratory Notebook (J. E. Cellis, ed., 1998) Academic Press: Animal Cell Culture (R. I. Freshney, ed., 1987): Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons: Methods in Enzymology (Academic Press, Inc.); Handbook of Experimental Immunology (D. M. Weir and CC. Blackwell, eds.), and “Methods and compositions for treating rheumatoid arthritis” WO2019209840A1, each of which is incorporated by reference herein. Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a pharmaceutical composition, and/or a method that “comprises” a list of elements (e.g., components, features, or steps) is not necessarily limited to only those elements (or components or steps), but may include other elements (or components or steps) not expressly listed or inherent to the pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”. It is understood that aspects and embodiments of the invention described herein include “consisting” and/or “consisting essentially of” aspects and embodiments.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

It will be further understood that there are a number of values disclosed therein, and that each value is also herein disclosed as “about” that particular value in addition to the value itself. In embodiments. “about” can be used to mean, for example, a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In various embodiments, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In an aspect, the disclosure provides pharmaceutical compositions comprising nicotinamide-riboside (NR) and pterostilbene (PT) in a ratio of between about 4:1 and 1:1, or between about 3:1 and 1.5:1, or about 2:1, effective derivatives thereof, and one or more pharmaceutically acceptable excipients or diluents.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia other generally recognized pharmacopoeia in addition to other formulations that are safe for use in animals, and more particularly in humans and/or non-human mammals.

As used herein the term “pharmaceutically acceptable diluent or excipient” or “pharmaceutically acceptable carrier” refers to an excipient, diluent, preservative, solubilizer, emulsifier, adjuvant, and/or vehicle with which nicotinamide-riboside (NR) and pterostilbene (PT) of the disclosure, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens: antioxidants such as ascorbic acid or sodium bisulfite: chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable diluent or excipient” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

Formulations of a pharmaceutical composition suitable for administration typically generally comprise the active ingredient combined with a pharmaceutically acceptable diluents or excipients, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Formulations may also include aqueous solutions which may contain excipients such as salts, carbohydrates and buffering agents or sterile, pyrogen-free, water. Exemplary administration forms may include solutions or suspensions in sterile aqueous solutions, for example, aqueous propylene glycol or dextrose solutions. Such dosage forms can be suitably buffered, if desired.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, and/or aromatic substances and the like which do not deleteriously interact with the formulation.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

In an aspect, the disclosure provides methods of treating or preventing a disease or disorder in a subject in need thereof, such as osteoarthritis and cartilage injury or degradation, comprising administering the pharmaceutical compositions of the disclosure to the subject. In some embodiments, the malignancy comprises osteoarthritis.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject.” “patient” or “individual” as used herein, includes any animal in need of being treated by the methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

In some embodiments, administering comprises administering a therapeutically effective amount to a subject.

As used herein, the term “amount” refers to “an amount effective” or “an effective amount” of a cell to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset: or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat.” “treatment.” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” is encompassed by “treatment” and may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

The pharmaceutical compositions of the disclosure may be administered in a number of ways depending upon whether local or systemic treatment is desired. The pharmaceutical compositions are typically suitable for parenteral administration, wherein administration includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intranasal, intratracheal, intraarterial, intrathecal, intradermal, intrasynovial injection or infusions. In some embodiments, the pharmaceutical compositions of the present disclosure comprise intravenous administration.

In some embodiments, the pharmaceutical composition comprising said compounds of the disclosure are administered in combination with a combination partner. The term “combination” refers to either a fixed combination in one dosage unit form, or a kit of parts for the combined administration where the pharmaceutical composition of the disclosure, and a combination partner (e.g., another drug as explained below, also referred to as “therapeutic agent” or “co-agent”) may be administered independently at the same time or separately within time intervals. In some circumstances the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

The term “derivative” refers to a chemical substance related structurally to another substance, or a chemical substance that can be made from another substance (i.e., the substance it is derived from), e.g., through chemical or enzymatic modification.

### EXAMPLES

### Example 1

### Oral Supplementation of Combination of Nicotinamide-Riboside (NR) and Pterostilbene (NRPT) Inhibits IL-1b Induced NO Release

Testing the effects of NR alone, PT alone and NR+PT (5:1, NR 0.2 mM and PT 0.04 mM) on chondrocyte catabolic responses to pro-inflammatory cytokine IL-1beta in primary human knee chondrocytes in vitro. It was determined that NR+PT showed stronger inhibitory effect compared to NR alone or PT alone, suggesting a synergistic effect of NR+PT combination in inhibiting IL-1beta-induced catabolic activities.

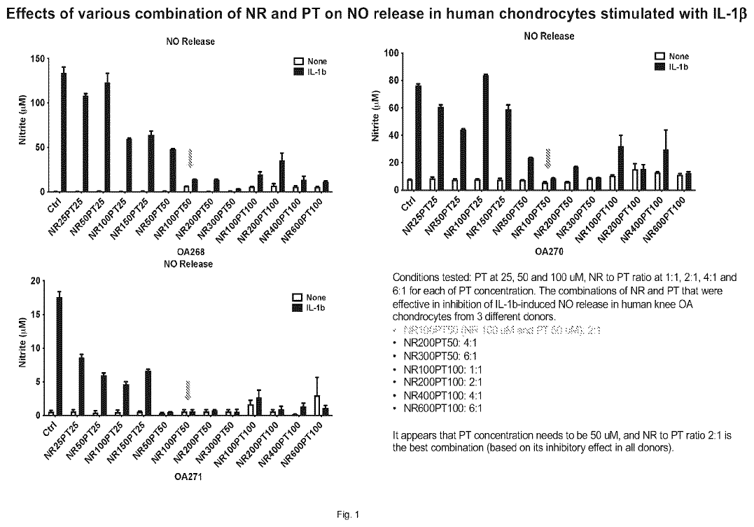

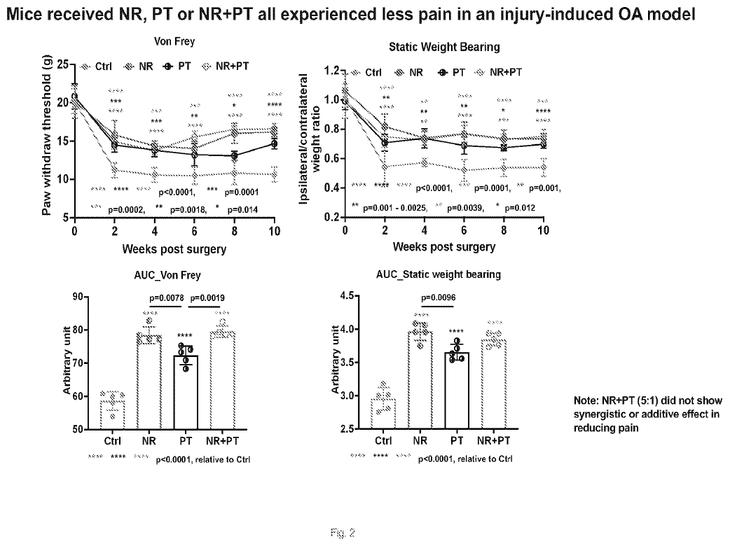

In vivo studies were designed to determine if oral supplementation of NR+PT (5:1) with NR at 400 mg/kg/day (the commonly used dose in animal studies) and PT at 80 mg/kg/day is better than NR alone (400 mg/kg/day) or PT alone (80 mg/kg/day), in limiting cartilage degradation and reducing pain in an injury-induced OA model. Pain behavior studies demonstrated that mice received NR alone, PT alone, and NR+PT combination all experienced significantly less pain compared to the non-treated controls. Notably, NR alone or NR+PT appeared to have a slightly better efficacy, but there is no statistical significance between NR and NR+PT groups. Because synergistic or additive effect of NR+PT combination was not initially observed, in vitro studies tested the combination of NR and PT at different ratio (NR:PT, 1:1, 2:1, 4:1, 6:1) and different concentrations (based on PT concentration at 25, 50 and 100 uM) on chondrocyte response to IL-1beta. Because IL-1b is known to induce nitric oxide (NO) production in chondrocytes, NO release (measured from the conditioned media) was used as a readout. It was determined that NR+PT at 2:1 ratio had the best efficacy in inhibition of IL-1b induced NO release, particularly for NR at 0.1 mM and PT at 0.05 mM. It appears that NR+PT at 2:1 ratio, a new formulation, could be more effective in suppression of OA development and pain. See, FIGS. 1 and 2.

### Experimental Design

C57BL/6J mice at 12 weeks of age were subjected to joint injury via destabilization of medial meniscus (DMM) surgery through transection of meniscotibial ligament. Sham surgery (just skin incision) was performed on separate group of mice. The day after DMM surgery, mice were divided into 4 groups and started to receive treatment as shown below via gavage daily.
1. Vehicle control
2. NR (400 mg/kg/day)
3. Pterostilbene (PT) (80 mg/kg/day)
4. NR+PT (400 mg/kg/day NR+80 mg/kg/day PT).

Pain-like behaviors was evaluated at baseline (1 weeks before the surgery) and 2, 4, 6, 8 and 10 weeks after DMM surgery.
1. Von Frey test (mechanical stimulus evoked nociception)
2. Static weight bearing (weight distribution asymmetry induced by pain).

### Example 2

### Oral Supplementation of Combination of Nicotinamide-Riboside (NR) and Pterostilbene (NRPT) Increases Circulation and Joint Tissue NAD⁺ Levels in Mice

NR was found to be rapidly degraded to NAM in gut and circulation. The short stability of NR in circulation could compromise the ability NR to reach peripheral tissues and limit its clinical application. A recent study demonstrated that conversion of NR to NAM was reduced 3-fold in the germ-free mice after oral intake. Since orally administered pterostilbene (PT) can modulate microbiota and exhibits anti-bacterial activity, this example sought to determine if blood and joint tissue NAD⁺ levels would be higher in mice that received a combination of NR and PT compared to NR alone. Metabolic tracing was performed using isotopically labeled NR, which incorporates a ¹³C-U-ribose scaffold and ¹³C, ¹⁵N, ¹⁸O amide on the nicotinamide ring to investigate how NR and/or its metabolites are utilized for NAD+ synthesis in circulation and joint tissues after mice orally intake of NR with and without PT. The labeled NRCl (M+8) was orally administered to mice via gavage at 100 mg/kg in the presence or absence of PT at 50 mg/kg (2:1 ratio) for 2 hours. The labeled and naturally occurring NR and major NAD+ metabolites such as NADH, NADP, NAM (nicotinamide), methyl NAM (Me-NAM) and methyl pyridone (Me-PY) were measured from whole blood and joint issues by LC-MS.

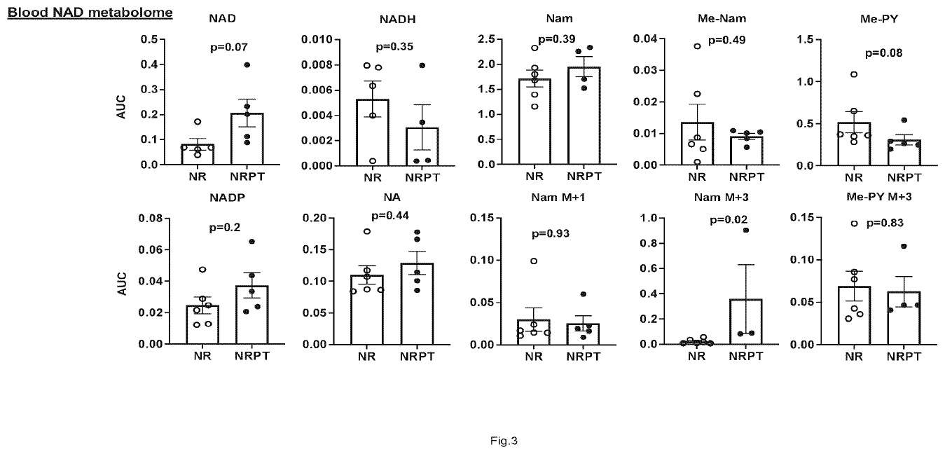

FIG. 3 shows tracing of isotopically labeled NR MW+8, ¹³C 5 ribose, ¹⁸O (amide), ¹⁵N (pyridine)) NR with and without PT. Blood samples for measurements by LCMS were collected 2 h after oral administration. Values are AUC normalized to volume (100 uL) and two internal standards (labeled riboside-4-pyridone carboxamide M+2 and Nam+4). Naturally occurring NAD, NADH, NADP, NAM, Me-NAM, Me-PY, and nicotinic acid (NA) were measured along with the isotopomers incorporating the isotopic tracers. While no NAD, NADH or NADP appear to incorporate the ¹³C-labeled ribose or ¹⁵N, ¹⁸O labeled nicotinamide scaffold in either treatment, in the NRPT treatment, PT affects how much labeled-nicotinamide (Nam+3) M+3 is available for NAD synthesis. Overall, NRPT appears to increase the Nam pool. PT also appears to protect NR from its degradation to NA, since no NA M+1 was detected and Nam M+3 levels were higher upon NRPT treatment than NR alone. Overall, NRPT treatment data show a trend towards a reduction in the levels of me-PY. In conclusion, PT in the NRPT formulation protects the nicotinamide of NR from its microbial conversion to NA.

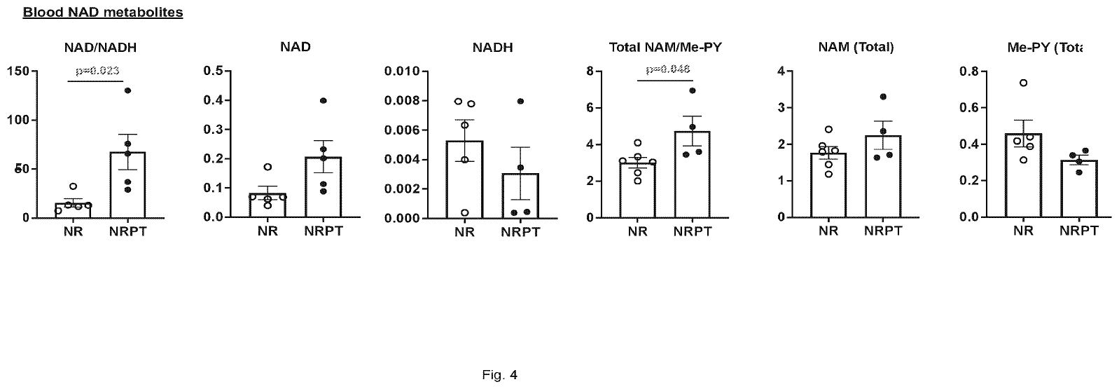

FIG. 4 shows the ratio of naturally occurring NAD, and NADH change in favor of NAD when NR is combined to PT. This change is statistically significant. The NRPT combination appears to favor NAD over NADH. This observation would indicate that NRPT provides means to reduce reductive stress and improve on blood bioenergetics. Similarly, the NAM/Me-PY levels are indicative of a less ROS-promoting environment when PT is present. Overall, NRPT also appears to increase the NAM pool available for NAD synthesis compared with NR only. In summary, PT in the NRPT formulation improves on the NAD/NADH ratio and total NAD levels in blood. The NRPT combination decreases Me-PY, a ROS and oxidative stress marker, considered by some as a uremic toxin.

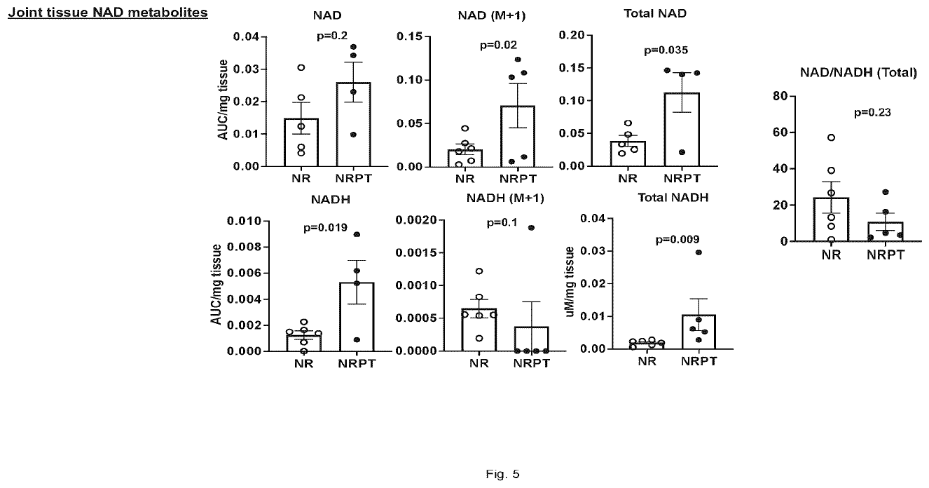

FIG. 5 shows tracing of isotopically labeled NR MW+8, ¹³C 5 ribose, ¹⁸O (amide), ¹⁵N (pyridine)) NR with and without PT. Knee joint tissue samples for measurements by LC-MS were collected 2 h after oral administration. Values are AUC normalized to protein content and two internal standards (labeled riboside-4-pyridone carboxamide M+2 and Nam+4). Naturally occurring NAD and M+1 NAD increase upon NRPT supplementation compared with NR only in the knee-joint tissues. Total NAD levels increase by more than 2 folds when NRPT is used instead of NR only. In this tissues the NADH also increases in the significant manner. Notably, more M+1 incorporation is observed in NR only than in NRPT supplementation in NADH, while more NAD M+1 is present in NRPT compared with NR only. This further supports that the NRPT combination increases NAD and NADH better than NR alone in a statistically significant manner. In conclusion, PT in the NRPT formulation enhances the total levels of NAD and NADH in the mice knee joint.

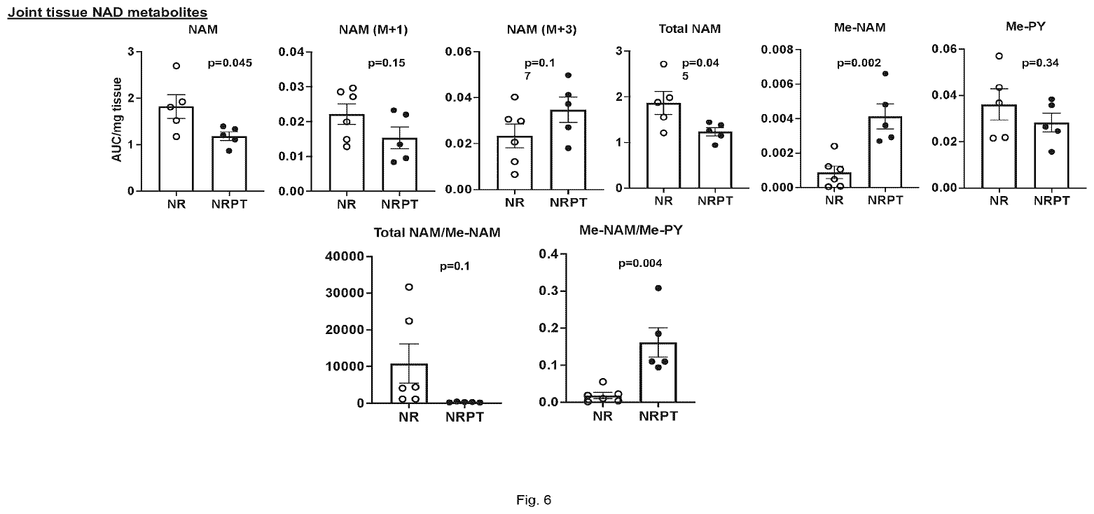

FIG. 6 shows tracing of isotopically labeled NR MW+8, ¹³C 5 ribose, ¹⁸O (amide), ¹⁵N (pyridine)) NR with and without PT. Knee joint tissue samples for measurements by LC-MS were collected 2 h after oral administration. Values are AUC normalized to protein content and two internal standards (labeled riboside-4-pyridone carboxamide M+2 and Nam+4). In knee joint tissues, the NRPT combination results in lower levels of free NAM. As for blood, there is more Nam M+3 than Nam M+1 available. Although Me-Nam is more abundant in NRPT joint tissues, oxidation of methyl-PY is less prominent compared to NR only. This observation could be an indication of reduced levels of ROS in NRPT-treated mice. In conclusion, NRPT in mice protects against the production of the uremic toxin Me-PY.

What is claimed is:

1. A method of treatment for osteoarthritis in a subject comprising administering to a subject in need an effective amount of a composition comprising nicotinamide-riboside (NR) and pterostilbene (PT) in a ratio of between about 4:1 and 1:1, or between about 3:1 and 1.5:1, or about 2:1.

2. The method of claim 1, wherein the NRH: PT ratio is about 2:1.

3. The method of claim 1, wherein the administration decreases nitric oxide (NO) levels in articular chondrocytes of the subject.

4. The method of claim 1, wherein the composition further comprises pharmaceutically acceptable excipient.

5. The method of claim 1, wherein the administration inhibits progression of osteoarthritis.

6. The method of claim 1, wherein the administration is oral.

7. The method of claim 1, wherein the administration is parenteral.

8. A method of treatment for cartilage injury in a subject comprising administering to a subject in need thereof an effective amount of a composition comprising nicotinamide-riboside (NR) and pterostilbene (PT) in a ratio of between about 4:1 and 1:1, or between about 3:1 and 2.5:1, or about 2:1.

9. The method of claim 8, wherein the NRH: PT ratio is about 2:1.

10. The method of claim 8, wherein the administration decreases nitric oxide (NO) levels in articular chondrocytes of the subject.

11. The method of claim 8, wherein the composition further comprises pharmaceutically acceptable excipient.

12. The method of claim 8, wherein the administration inhibits progression of osteoarthritis.

13. The method of claim 8, wherein the administration is oral.

14. The method of claim 8, wherein the administration is parenteral.

15. A pharmaceutical composition comprising nicotinamide-riboside (NR) and pterostilbene (PT) in a ratio of between about 4:1 and 1:1, or between about 3:1 and 1.5:1, or about 2:1.
